(19) United States
(12) Patent Application Publication
(10) Pub. No.: US 2014/0018750 A1
(43) Pub. Date: Jan. 16, 2014

(54) METHOD AND DEVICE OR PHARMACEUTICAL COMPOSITIONS FOR THE TRANSDERMAL DELIVERY OF MAGNESIUM DIRECTLY TO THE NEUROMUSCULAR JUNCTION FOR THE TREATMENT OF MUSCLE CRAMPING

(71) Applicants: Michael J. Pagliaro, White Plains, NY (US); Marie M. Pagliaro, White Plains, NY (US)

(72) Inventors: Michael J. Pagliaro, White Plains, NY (US); Marie M. Pagliaro, White Plains, NY (US)

(21) Appl. No.: 13/919,407

(22) Filed: Jun. 17, 2013

Related U.S. Application Data
(60) Provisional application No. 61/669,933, filed on Jul. 10, 2012.

(51) Int. Cl. A61K 9/70; A61K 9/00; A61M 35/00; A61K 33/06

(52) U.S. Cl. CPC A61K 9/703; A61K 33/06; A61K 9/0014; A61M 35/00
USPC 604/290; 424/682; 424/443

(57) ABSTRACT

A method for treating muscle cramping (e.g., acute nocturnal) includes the steps of placing a transdermal delivery agent on skin above an affected muscle that is in spasm. The transdermal delivery agent includes a composition that includes a therapeutic amount of magnesium. Placement of the transdermal delivery agent on the skin results in transdermal delivery of the magnesium directly to the affected muscle that is in spasm, thereby providing therapeutic relief while limiting the delivery of the magnesium to the entire circulatory system (systemic effect).

### CROSS REFERENCE TO RELATED APPLICATION

The present application claims priority to U.S. patent Application No. 61/669,933, filed Jul. 10, 2012, which is hereby incorporated by reference in its entirety.

### TECHNICAL FIELD

This invention relates generally to methods, devices and pharmaceutical compositions for applying magnesium to relieve muscle cramping at the onset and during such attack.

### BACKGROUND OF THE INVENTION

Many people suffer from painful spontaneous muscle cramping, particularly while sleeping and following physical exertion. The elderly are particularly prone to suffer from painful leg cramps. Many different preventative measures have been suggested for nocturnal leg cramps, including stretching devices (see, e.g., U.S. Pat. Nos. 6,966,883 and 6,027,434), an electrical stimulation device (see, e.g., U.S. Pat. No. 5,759,198), the use of a heating pad on the legs before bed, dietary changes and supplements, quinine sulfate tablets, drinking tonic water as a source of quinine, regular exercise, placing a bar of Ivory® Soap under the sheets, and likely many more. However, all of these proposed remedies have been found to be less than optimal. None of the above modalities is designed to ameliorate an acute muscle spasm once it has begun.

Delivery of drugs by the transdermal route has been known to be possible for many years. Creams, gels, ointments and lotions have been used for more than a century. The earliest patented transdermal devices were medicated bandages, usually with the drug mixed into the adhesive, that were designed to bring a known quantity of drug to a known area of skin for a known time. Controlled release transdermal devices were then developed to provide delivery of a drug to the skin for a prolonged period of time, generally a day, several days, or a week. Although transdermal patches have been developed for many different treatments generally to achieve therapeutic serum levels (e.g. estrogen, narcotic analgesics, scopolamine etc.), the inventor is not aware of any transdermal patch or gel for use in treating acute nocturnal muscle cramping and sports injury.

### SUMMARY

The present invention provides a method and device or pharmaceutical compositions for transdermally applying magnesium to the leg or other affected area to directly transport magnesium to the neuromuscular junction thereby overcoming the limitations of prior art that merely seek to prevent spasm. The current invention rapidly relieves acute nocturnal leg cramping, sports and other muscle injuries. In accordance with one embodiment, the present invention provides relief of an acute night leg cramp within a predetermined time period which can be on the order of less than 10 minutes; less than 5 minutes, etc.

Unlike traditional transdermal products which are systemic in nature and deliver drug into the patient's circulatory system for treating a condition, the method and device of the present invention is directed to treating a muscle that is currently in spasm without acting as a systemic delivery system. Instead, the method and device of the present invention is for immediate local treatment of a muscle in spasm (acute cramping) by providing localized treatment (non-systemic in nature).

### BRIEF DESCRIPTION OF THE DRAWING FIGURES

FIG. 1 is a cross-sectional view of one exemplary transdermal patch in accordance with the present invention for transdermally applying magnesium sulfate to target tissue, such as the leg muscles.

### DETAILED DESCRIPTION OF CERTAIN EMBODIMENTS

The preferred embodiment of the present invention is a method and device or pharmaceutical composition for transdermally applying magnesium sulfate to one or muscle that is in spasm (i.e., undergoing cramping) and in particular, is intended for application to a leg muscle that is undergoing acute cramping. In particular, one preferred embodiment corresponds to the use of a transdermal patch or gel to apply a magnesium sulfate solution/gel to muscles, particularly but not limited to leg muscles, when awoken from sleep by an acute episode or immediately after muscle injury to treat muscle cramping or spasm. Thus, unlike traditional products that are applied proactively to prevent such episodes, the present invention is directed to a method of treatment and a device for treating a muscle that is currently in spasm for immediate relief thereof.

The present invention is thus also directed to a transdermal patch or gel which can have a conventional structure; however, in accordance with the present invention is specifically adapted to apply magnesium sulfate in an appropriate amount (a therapeutic amount which is an amount that is sufficient to provide for relief of pain) to relieve muscle cramping. Such transdermal patch may be used at the time of the attack or be worn only while sleeping, or could also be worn at other times or at all times, in the event that the user suffers from muscle cramping during waking hours. However, the present invention is particular suitable for localized treatment of a muscle that is in spasm.

The following examples are illustrative of one preferred formulation of the invention. All percentages are based on the percent by weight (e.g. by molecular weight) of the formulation prepared unless otherwise indicated and all the totals equal 100% by weight.

| | % (M/W) |
|---|---|
| Material | (Molecular Weight) |
| MAGNESIUM SULFATE USP | 8.000 |
| PURIFIED WATER USP, EP | 76.340 |
| GLYCEROL (VEG. ORIG.) 99.5% USP, EP | 12.900 |
| NATROSOL 250 HHR | 2.500 |
| METHYL HYDROXYBENZOATE NF, EP | 0.180 |
| SORBIC ACID NF, EP | 0.080 |
| TOTALS | 100% |

The follow examples set forth alternative formulations. All values are given as weight percent by molecular weight.

| | For- | For- | For- | For- | For- |
|---|---|---|---|---|---|
| Material | mula A | mula B | mula C | mula D | mula E |
| Magnesium Sulfate USP | 8.00 | 8.00 | 8.00 | 8.00 | 8.00 |
| Pemulen TR-1 NF | 0.10 | 0.50 | 0.25 | 1.00 | 0.30 |
| Carbopol ® 974P* | 1.00 | 1.00 | 1.50 | 1.00 | 1.50 |
| Natrosol 250 HHX | 2.00 | 2.00 | 2.00 | 2.00 | 2.00 |
| Glycerol USP/BP | 12.9 | 14.6 | 12.9 | 12.9 | 12.9 |
| Sorbic acid NF/EP | 0.08 | 0.80 | 0.08 | 0.08 | 0.08 |
| Methyl Hydroxybenzoate | 0.18 | 0.18 | 0.18 | 0.18 | 0.18 |
| NF, EP | | | | | |
| Purified water USP/EP | 75.74 | 72.92 | 75.09 | 74.84 | 75.04 |
| Total Weight | 100 | 100 | 100 | 100 | 100 |

It will be appreciated that the above formulations are merely exemplary and not limiting of the present invention. With respect to the above formulations, one of skill in the art will appreciate that the Natrosol component is a hydroethylcellulose product that is commercially available from Ashland, Inc. The Natrosol component can be used as a high efficiency nonionic thickener, water retention aid and rheology modifier. Pemulen TR-1. NF is a high molecular weight copolymer of acrylic acid and a long chain alkyl methacrylate crosslinked with allyl ethers of pentaerythritol and is commercially available from Lubrizol.

In particular and according to one embodiment, an aqueous magnesium sulfate transdermal gel impregnated patch is provided and is formed using standard GRAS (generally recognized as safe) ingredients such as propylene glycol, glycerin, polyacryclic acid, etc. as known to those of ordinary skill in the art. An appropriate amount of magnesium sulfate, e.g., about 8% (MW), is included within the gel or other material that is contained in the patch in order to provide the desired therapeutic effect. In other words, a therapeutic amount of the agent (magnesium sulfate) is included within the composition that is included within the patch and is delivered to the patient by a transdermal delivery mechanism. It will be understood that the exact amount chosen is the amount necessary to provide proper relief from muscle cramping without excess exposure to magnesium sulfate. The amount is thus an amount that provides the desired therapeutic effects (i.e., the relief of pain at the target site) as described herein, while at the same time does not result in the patient experiencing undesirable side effects. More specifically, the therapeutic amount is an amount that produces immediate (preferably, less than 10 minutes and more preferably, less than or about 5 minutes), localized relief (therapeutic relief) to the muscle in spasm (muscle cramp) in contrast to providing a systemic effect by being absorbed into the circulatory system. The present invention achieves these results by delivering the magnesium to the underlying target tissue, in this case, the underlying muscle that is in spasm.

In one embodiment, the composition includes a water-swellable, water-insoluble, cross-linked polyacrylic acid polymer (polyacrylate polymer) which cross-linked with allyl sucrose or allyl pentaerythritol. Any number of commercially available polyacrylate polymers can be used including by not limited to: Carbopol® 974 polymer; Carbopol® 940 polymer; Carbopol® 941 polymer, and Carbopol® 981 polymer, all of which are commercially available from a number of different suppliers.

The structure of the patch is conventional, chosen to provide a transfer of an amount of the magnesium sulfate transdermal gel formulation to the neuromuscular junction (point on pain) of muscles susceptible to cramping which provides relief from such cramping. One type of conventional patch structure is disclosed in U.S. Pat. No. 4,597,961, which is hereby incorporated in its entirety by reference. The '961 Patent discloses a transdermal patch for the application of nicotine to the body. Another type of conventional patch structure is that used in the BENGAY® Pain Relieving Patch. There are many other types of transdermal patches known to those of ordinary skill in the art, and one of ordinary skill in the art will readily recognize that the patch structure simply needs to be able to allow the magnesium sulfate solution/gel to contact the skin in appropriate amounts (therapeutic amounts) to facilitate the transfer through the skin to the neuromuscular junction.

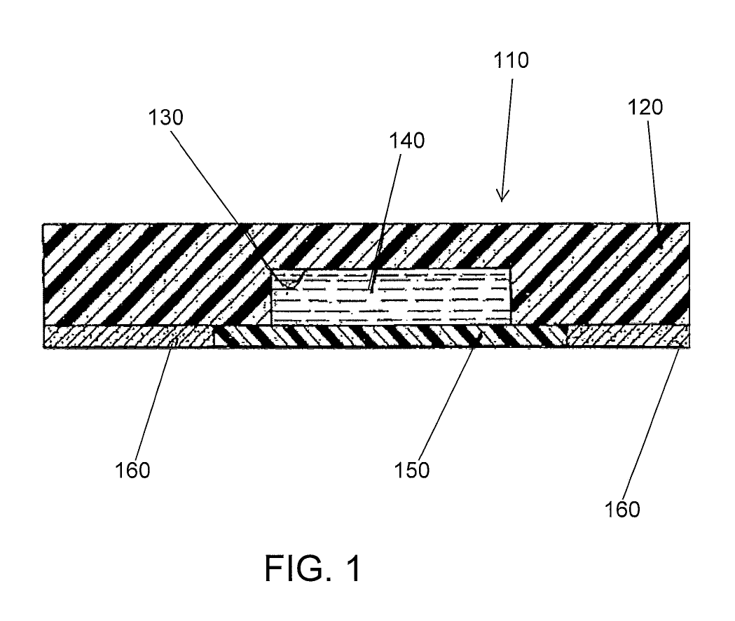

FIG. 1 illustrates an exemplary transdermal patch construction and in particular, transdermal patch 100 is formed of a body 110 that has a width and thickness that are suitable for the intended application. The body 110 has a backing 120 that is impermeable relative to the active agent to be delivered (in this case, magnesium sulfate) and can be formed of any number of different materials, including but not limited to flexible and impervious plastic or rubber materials (e.g., polyvinylidene chloride, polyethylene, polypropylene, nylon, silicon rubber, etc. The body 110 includes a reservoir or depot (cavity) 130 that holds the therapeutic composition that is to be delivered to the person. The reservoir 130 can thus be a recessed opening that is formed along one face (surface) of the body 110 as shown. The dimensions and shape of the reservoir 130 vary depending upon the precise application. Within the reservoir 130, the therapeutic composition 140 of the present invention is disposed for delivery to the person. As mentioned above, the amount and the state of the therapeutic composition 140 can vary depending upon the particular application; however, the amount is a therapeutic amount that provides the desired, intended therapeutic benefits that are disclosed herein (i.e., provide immediate relief to the underlying muscle that is in spasm (cramp).

A membrane 150 is provided for sealing the therapeutic composition 140 within the reservoir 130. The membrane 150 thus covers the reservoir 130. The membrane 150 can be formed of any number of different materials so long as the layer 150 functions as a membrane in that it allows the controlled release of the composition 140 therethrough (e.g., through diffusion through the membrane or through erosion of the membrane, etc.). Some suitable materials for the membrane 150 include but are not limited to suitable microporous and flexible plastic or rubber that is inert to the composition 140. The membrane 150 is thus an active-agent release membrane.

A suitable adhesive structure (i.e., a contact adhesive layer) 160 is provided on at least select areas of the backing 120 to provide a means for attaching the patch 100 to the patient's skin. The adhesive structure 160 can thus be provided on either side of, or completely around the membrane 150.

In accordance with one embodiment of the present invention, the magnesium sulfate transdermal gel formulations (composition 140) are chosen to have the following characteristics: 1) onset of action (therapeutic effect/therapeutic relief) within about one to at most two minutes with more complete therapeutic relief being felt in preferably less than 10 minutes, more preferably, less than 5 minutes; 2) therapeutic levels at the site of action, the underlying musculature, without the need for high systemic levels of magnesium (since the delivery mechanism is not systemic in nature and does not rely on the patient's circulatory system); 3) serum magnesium levels should be no greater than baseline within about an hour to avoid the need for a high degree of kidney clearance which is particularly important in the elderly who may have impaired renal function; and 4) avoidance of gastrointestinal side effects as magnesium salts and specifically magnesium citrate, magnesium hydroxide and magnesium sulfate are commonly used as saline laxatives. High local concentrations combined with low systemic levels achieved with a transdermal formulation ensure maximum therapeutic effect at the site of action, the neuromuscular junction, and minimal systemic and gastrointestinal adverse events. This is in contrast to conventional transdermal drug delivery products which are constructed to provide systemic effects by having drug dosages and formulations which result in the drug being delivered into the circulatory system of the patient, thereby acting in a systemic manner.

It will thus be appreciated that the above features are merely exemplary of one embodiment and the composition 140 can have other characteristics. For example, the onset of the therapeutic effect can be within about 5 minutes (less than 10 minutes) in one embodiment. In addition, as discussed herein, the use of a patch structure is merely one means for delivering magnesium in a transdermal nature and there are other delivery means for delivering magnesium, such as application of a gel, etc.

Magnesium sulfate (Epsom salt), is regulated in two distinct ways in the United States. First, it is regulated as a drug under the OTC (over the counter) laxative monograph. Second, it is regulated in a “limbograndfathered” OTC category as a soaking aid for minor sprains and bruises. Magnesium sulfate is not included in an OTC drug monograph for this latter use. Thus, it is simultaneously a drug when used internally as a laxative and a general health product when used for soaking. In the United States, magnesium sulfate U.S.P. (heptahydrate), Epsom salt, may be marketed in the U.S. in a patch formulation without prior FDA approval. An Epsom salt patch thus falls under the existing OTC system. Current labeling of Epsom salt products in the second category includes the indication for external use as a soaking aid for minor sprains and bruises.

Magnesium sulfate U.S.P. heptahydrate MgS0407H2O (Epsom salt) exists as colorless crystals or white powder that is freely soluble in water −71 g/100 ml at 20 degrees C. For external use, current OTC labeling of Epsom salt products calls for the dissolution of two cupfuls of Epsom salt in a gallon of water. This corresponds to a solution of roughly 6.2% magnesium sulfate.

It is well known that local magnesium depletion leads to increased neuronal excitability and enhanced neuromuscular transmission with symptoms that include tremor, ataxia, tetany and cramps. Magnesium is believed to have a curare-like action at the neuromuscular junction as it interferes with the release of acetylcholine from motor nerve terminals thereby inhibiting muscle contraction and relieving cramps. However, the action of magnesium at the neuromuscular junction can be blocked by calcium.

Transdermally applied magnesium readily penetrates the skin and enters the underlying microvasculature producing high concentrations of magnesium in the muscle while minimizing systemic absorption. Accordingly, the magnesium is delivered directly to the target location (underlying muscle that is in spasm) to produce localized, immediate relief and not systemic benefits (since the goal is not to deliver magnesium into the patient's circulatory system). Unlike an Epsom salt bath in which the patient's entire body or body part is submerged in the bath, only a small portion of the body surface is exposed to magnesium when applied transdermally, e.g., using a transdermal patch. Magnesium is not metabolized and magnesium is excreted solely by the kidney at a rate proportional to the serum concentration and the glomerular filtration rate.

Magnesium sulfate is rapidly and extensively absorbed through the skin and thus, the present system and device is able to provide immediate, localized relief to a muscle that is in spasm. The present Applicant has discovered that the extent and speed of absorption can be further enhanced, for example, using higher concentrations of magnesium sulfate. Further, the addition of a small quantity of one or more GRAS (Generally Recognized as Safe) skin penetration enhancers e.g., Sodium Lauryl Sulfate, propylene glycol, isopropyl myristate, ethanol, DMSO, etc., helps ensure rapid skin permeation with high local muscle concentrations and minimum systemic concentrations of magnesium.

Based on the foregoing, one embodiment of the present invention is an 8% magnesium sulfate aqueous transdermal gel composition (composition 140) that is incorporated into a patch (e.g., patch 100) of approximately 10 cm×14 cm. Although one of ordinary skill in the art will readily recognize that other formulations of magnesium sulfate and other patch sizes are also well within the scope of the present invention, so long as the combined magnesium sulfate solution/gel and transdermal patch allow the transfer of an appropriate amount of magnesium sulfate through the skin to provide relief from muscle cramping, without excess exposure to magnesium sulfate as would occur in a systemic type treatment.

Although this disclosure discusses both solutions and gels of magnesium sulfate, one of ordinary skill in the art will readily recognize that the two terms are essentially interchangeable, and a solution or gel can either be alternatively used or the more appropriate one be chosen depending on the type of patch selected.

Although the preferred embodiment of the present invention uses a transdermal patch for applying a solution/gel having a magnesium salt (preferably magnesium sulfate) to the leg muscles, in alternative embodiments the solution/gel prepared in the manner as discussed above may be applied directly to the skin via a gel, ointment, lotion, cream or spray, instead of via a transdermal patch. In other words, the magnesium based composition can be applied directly to the skin of the patient over the muscle that is in spasm. The delivery of the magnesium through the skin to the underlying muscle tissue in spasm (i.e., to the neuromuscular junction of the affected muscle) provides relief to the patient.

In accordance with the present invention, the transdermal patches disclosed herein are specifically configured in view of the actual pathophysiology of muscle spasms. A spasm occurs because there is a deficiency of divalent cations, specifically magnesium, at the neuromuscular junction which results in tetanic contractions of the muscle. As is known, the neuromuscular junction connects the nervous system to the muscular system via synapses between efferent nerve fibers and muscle fibers, also known as muscle cells. Traditionally, magnesium supplementation is used to increase systemic levels and ultimately deliver sufficient magnesium to the neuromuscular junction. However and due to the fact that such supplementation is systemic in nature, the supplementation is delivered over the entire body through the patient's circulatory system. In contrast, the patches made in accordance with the present invention deliver magnesium directly to the neuromuscular junction without the need to create high systemic concentrations.

In addition, while 8% magnesium, by weight, is efficacious, it will be appreciated that values of less than 8% by weight can also be suitable in given applications. For example, magnesium may be present in a range of between about 4% to 8%, by weight.

### EXAMPLES

A person suffering from acute nocturnal muscle cramping used a composition (e.g., a composition including about 8% by MW of magnesium sulfate) made in accordance with the present invention and experienced quick relief from the pain associated with the cramping. The composition was delivered to the local area (e.g., leg) that was experiencing the pain (due to ongoing muscle spasm) and immediate relief (within 5 minutes) was observed by the patient. As a result, a therapeutic effect was observed and documented by such person.

Patient Doris suffering from reoccurring severe muscle cramps in the right foot applied a liquid solution part of which was 8% by MW of magnesium sulfate to the area and experienced relief within three minutes.

Patient Norma who experienced severe leg cramps in conjunction with restless leg syndrome nightly soaked a towel in composition which included 8% by MW of magnesium sulfate, applied the towel to the affected area and experienced relief from both symptoms within four minutes.

Patient Henry suffered from severe muscle cramps in the upper right arm as a result of exertion applied a liquid form of 8% by MW of magnesium sulfate directly to the affected area and began to experience relief within three minutes.

Patient Michael who suffered from sciatica applied a cream composed of aloe combined with 8% by MW of magnesium sulfate to the sciatic nerve on the buttock and experienced relief within six minutes.

It will be appreciated that when the present formulation is embodied in a patch, the patch needs only to be in place until the muscle spasm is relieved or until a predetermined period of time passes (e.g., 30 minutes). In the event of using a gel or cream, the cream/gel disappears naturally during use.

1. A method for treating muscle cramping comprising the step of:
placing a transdermal delivery agent on skin above an affected muscle that is in spasm, wherein the transdermal delivery agent includes a composition that includes a therapeutic amount of magnesium, wherein placement of the transdermal delivery agent on the skin results in transdermal delivery of the magnesium directly to the affected muscle that is in spasm, thereby providing therapeutic relief.

2. The method of claim 1, wherein the transdermal delivery agent comprises a transdermal patch that includes a reservoir that contains the composition and a membrane covering the reservoir and constructed such that the magnesium passes therethrough.

3. The method of claim 1, wherein the therapeutic amount of magnesium is selected such that therapeutic relief is observed within about 5 minutes from placing the transdermal delivery agent on the skin.

4. The method of claim 1, wherein the magnesium in the magnesium containing composition comprises magnesium sulfate.

5. The method of claim 4, wherein magnesium sulfate is present in an amount of about 8% by weight (MW) of the total magnesium containing composition.

6. The method of claim 1, wherein the magnesium containing composition is selected from the group consisting of an aqueous transdermal gel, an aqueous transdermal lotion, an aqueous transdermal ointment, and an aqueous transdermal cream.

7. The method of claim 1, wherein the magnesium containing composition further includes one or more ingredients selected from the group consisting of: propylene glycol and glycerin polyacrylic acid.

8. The method of claim 1, wherein the magnesium containing composition includes a waterswellable, water-insoluble, cross-linked polyacrylic acid polymer.

9. The method of claim 8, wherein the water-swellable, water insolublecross-linked polyacrylic polymer, is cross linked with allyl sucrose or allylpentaerythritol.

10. The method of claim 1, wherein the magnesium containing composition includes a penetration enhancing agent selected from the group consisting of sodium lauryl sulfate, propyleneglycol, isopropyl myristate, and ethanol.

11. The method of claim 1, wherein the magnesium containing composition by weight percent based on a total weight of the magnesium containing composition comprises:
(a) about 8.00% magnesium sulfate;
(b) about 76.34% purified water;
(c) about 12.90% glycerol;
(d) about 2.50% hydroethylcellulose;
(e) about 0.18% methyl hydroxybenzoate; and
(f) about 0.08% sorbic acid.

12. The method of claim 1, wherein the therapeutic amount of magnesium is selected such that it provides therapeutic levels locally at the affected muscle but in which serum magnesium levels are no greater than a baseline of the user within 2 hours.

13. The method of claim 1, wherein the transdermal delivery agent is constructed for direct delivery of magnesium to a neuromuscular junction of the affected muscle.

14. The method of claim 1, wherein the transdermal delivery agent is constructed to produce a high local concentration of magnesium at the site of the neuromuscular junction to maximize efficacy and produce low systemic concentration to minimize side effects.

15. The method of claim 1, wherein the muscle cramping is due to an acute nocturnal muscle cramping condition.

16. A method for treating and providing relief of muscle cramping comprising the step of:
delivering a therapeutic amount of magnesium to a neuromuscular junction of an affected muscle that is in spasm by placing a magnesium containing composition on skin above the affected muscle that is in spasm, the magnesium being delivered through the skin to the neuromuscular junction such that an onset of relief begins within about 5 minutes of placement of the magnesium containing composition on the skin due to therapeutic levels of magnesium being present at the affected muscle within the about 5 minutes, wherein the magnesium is present in an amount of about 8% by weight (MW) of the total magnesium containing composition.

17. The method of claim 16, wherein the magnesium containing composition is incorporated into a transdermal patch that includes:
a backing that has a reservoir formed therein, wherein the magnesium containing composition is disposed within the reservoir; and
a membrane covering the reservoir and constructed for placement on skin above the affected muscle to allow passage of the magnesium therethrough.

18. The method of claim 16, wherein the therapeutic amount of magnesium is selected such that serum magnesium levels are no greater than a serum magnesium baseline within about an hour after placement of the magnesium containing composition.

19. The method of claim 16, wherein the magnesium containing composition by weight percent based on a total weight of the magnesium containing composition comprises:
(g) about 8.00% magnesium sulfate;
(h) about 76.34% purified water;
(i) about 12.90% glycerol;
(j) about 2.50% hydroethylcellulose;
(k) about 0.18% methyl hydroxybenzoate; and
(l) about 0.08% sorbic acid.

20. A composition, by weight percent based on a total weight of the composition, for providing immediate relief of a muscle cramping condition comprising:
(a) about 8.00% magnesium sulfate;
(b) about 76.34% purified water;
(c) about 12.90% glycerol;
(d) about 2.50% hydroethylcellulose;
(e) about 0.18% methyl hydroxybenzoate; and
(f) about 0.08% sorbic acid.
